(19) United States
(12) Patent Application Publication
(10) Pub. No.: US 2004/0043403 A1
(43) Pub. Date: Mar. 4, 2004

(54) Process for determining the biological activity of epidermal growth factor receptor tyrosine kinase inhibitors

(76) Inventors: Peter Furst, Rheinfelden (CH); Rita Grossenbacher, Moehlin (CH)

Correspondence Address: THOMAS HOXIE, NOVARTIS, CORPORATE INTELLECTUAL PROPERTY, ONE HEALTH PLAZA 430/2, EAST HANOVER, NJ, 07936-1080, US

(21) Appl. No.: 10/450,801

(22) PCT Filed: Dec. 18, 2001
(86) PCT No.: PCT/EP01/14927

(30) Foreign Application Priority Data
Dec. 20, 2000 (GB) 0031080.5

(51) Int. Cl. C12Q 1/68; A61K 31/519

(52) U.S. Cl. 435/6; 514/265.1

(57) ABSTRACT

The present invention relates to processes for determining the biological activity of compounds that inhibit the tyrosine kinase activity of the Epidermal Growth Factor Receptor (EGFR) aqnd to the use of transcription or translation products of genes the expression levels of which correlate with the biological activity of an EGFR tyrosine kinase inhibitor for determining the biological activity of such an EGFR tyrosine kinase inhibitor.

The present invention relates to processes for determining the biological activity of compounds that inhibit the tyrosine kinase activity of the Epidermal Growth Factor Receptor (EGFR) (hereinafter called “EGFR tyrosine kinase inhibitors”) and to the use of transcription or translation products of genes the expression levels of which correlate with the biological activity of an EGFR tyrosine kinase inhibitor for determining the biological activity of such an EGFR tyrosine kinase inhibitor.

Inhibitors of the tyrosine kinase activity of the EGFR, such as the pyrrolo-pyrimidine derivatives described in the European patent EP 0 682 027 B1 and the International Applications WO 97/02266 and WO 98/07726, have been shown to exhibit anti-proliferative activity. WO 97/02266 also discloses the pyrrolo-pyrimidine derivative (R)-6-(4-hydroxy-phenyl)-4-[(1-phenyl-ethyl)-amino]-7H-pyrrolo[2,3-d]pyrimidine (hereinafter referred to as COMPOUND A) which is a potent and selective inhibitor of the EGFR tyrosine kinase and exhibits anti-proliferative activity against Epidermal Growth Factor (EGF)-responsive tumors.

Due to the pharmacologically useful properties of compounds that inhibit the tyrosine kinase activity of the EGFR, there is a need to identify surrogate markers correlating with the biological activity of said inhibitors. In accordance with the present invention it has now surprisingly been found that the expression level of certain genes, such as the clusterin gene correlates with the biological activity of EGFR tyrosine kinase inhibitors such as COMPOUND A.

### DESCRIPTION OF THE FIGURES

FIGS. 1A and 1B show clusterin expression levels measured by real time Reverse Transcriptase Polymerase Chain Reaction (RT-PCR) in A431 cells upon COMPOUND A treatment. FIG. 1A: dose response; A431 cells are treated with 100 nM, 300 nM and 3 μM of COUMPOUND A for 16 or 24 h. FIG. 1B: time dependency; A431 cells are treated for 4 to 24 h with 300 nM or 3 μM of COMPOUND A.

On the Y axis fold expression relative to Dimethyl Sulfoxide (DMSO) control is plotted. Error bars=Standard Deviation (SD) (n=3).

FIG. 2 shows the dose response of clusterin induction in A431 cells upon COMPOUND A exposure.

A431 cells are treated with 10 nM to 1 μM of COMPOUND A or 0.03% DMSO for 24 h. Cell lysates are probed with anti-clusterin antibody (or with anti-actin antibody for control blots) by Western blotting. 43 kilo Dalton (kDa) signals represent a non-glycosylated form of clusterin and 60 kDa signals the glycosylated precursor. Lanes 1-5: 1 μM, 300 nM, 100 nM, 30 nM, and 10 nM of COMPOUND A, respectively; lane 6: 0.03% DMSO.

FIG. 3 shows the time course of clusterin induction in A431 cells upon COMPOUND A exposure.

A431 cells are treated with 300 nM of COMPOUND A for 32 h (lane 1), 24 h (lane 2), 16 h (lane 3), 8 h (lane 4), or 4 h (lane 5) or with 0.03% DMSO for 24 h (lane 6) or 4 h (lane 7). Cell lysates are probed with anti-clusterin antibody (or with anti-actin antibody for control blots) by Western blotting. 43 kDa signals represent a non-glycosylated form of clusterin and 60 kDa signals the glycosylated precursor.

FIG. 4 shows the relationship between clusterin induction by COMPOUND A and EGFR status.

A431 (lanes 1-2), MDA-MB468 (lanes 3-4), NCI-H596 (lanes 5-6), and NCI-H520 (lanes 7-8) are treated with 300 nM of COMPOUND A (lanes 2, 4, 6, and 8) or with 0.03% DMSO (lanes 1, 3, 5, and 7) for 24 h each. Cell lysates are probed with anti-clusterin antibody by Western blotting. 43 kDa signals represent a non-glycosylated form of clusterin and 60 kDa signals the glycosylated precursor. EGFR status: A431 (++++); MDA-MB468 (++++); NCI-H596 (++); and NCI-H520 (−).

### DETAILED DESCRIPTION OF THE INVENTION

The present invention provides genes, such as the clusterin gene, the expression levels of which correlate with the biological activity of EGFR tyrosine kinas inhibitors, such as COMPOUND A, which makes thes genes useful as surrogate markers for the biological activity of said tyrosine kinase inhibitors. Such surrogate markers or biomarkers can be used for example in clinical settings to optimize the use such as e.g. dosing and scheduling of EGFR tyrosine kinase inhibitors.

A gene the expression level of which correlates with the biological activity of an EGFR tyrosine kinase inhibitor can be expressed by the same cells which express EGFR and the expression level of such a gene can thus be measured in any cell line which expresses EFGR, in particular in cell lines of epithelial origin such as for example A431, or in any tissue expressing EGFR, in particular in tumor tissue, in skin, in hair bulbs and in mucosa of the mouth.

As another possibility, a gene the expression level of which correlates with the biological activity of an EGFR tyrosine kinase inhibitor can be expressed by cells other than that which express EGFR as part of a paracrine action. The expression level of such a gene can therefore be measured either in the same tissue which contains both cells which express said gene and cells which express EGFR or in a tissue or cells which only express(es) the mentioned gene but not the EGFR.

A gene the expression level of which correlates with the biological activity of an EGFR tyrosine kinase inhibitor can code for example for a secreted extracellular protein and its level of expression can therefore be measured for example on the basis of the amount of protein present in e.g. the interstice such as for example the plasma or in the cell supernatant.

The level of expression of a gene the expression level of which correlates with the biological activity of an EGFR tyrosine kinase inhibitor can be measured by any technical means on the basis of e.g. RNA expression using for example the technique of RT-PCR or on the basis of e.g. protein expression using for example the technique of Western blotting, immunohistochemistry or ELISA.

The invention relates to a process for determining the biological activity of a compound that inhibits the tyrosine kinase activity of the epidermal growth factor receptor (EGFR), which process comprises detecting in a biological sample the level of expression of a gene the expression level of which correlates with the biological activity of said EGFR tyrosine kinase inhibitor, said biological sample having been exposed to said EGFR tyrosine kinase inhibitor.

The invention also relates to a process for determining the biological activity of a compound that inhibits the tyrosine kinase activity of the EGFR, which process comprises exposing EGFR expressing cells of a mammal to an EGFR tyrosine kinase inhibitor and detecting ex vivo the level of expression of a gene the expression level of which correlates with the biological activity of said EGFR tyrosine kinase inhibitor.

In a preferred embodiment of the process of the preceding paragraph, EGFR expressing cells from a mammal are exposed to an EGFR tyrosine kinase inhibitor by administering such an inhibitor or a pharmaceutically acceptable salt thereof to said mammal.

In a preferred embodiment of the process of the present invention, the anti-proliferative activity, especially the anti-tumor activity, of an EGFR tyrosine kinase inhibitor is determined.

The invention further relates to the use of a transcription or translation product of a gene the expression level of which in a mammalian cell correlates with the biological activity of an EGFR tyrosine kinase inhibitor for determining ex vivo the biological activity of such an EGFR tyrosine kinase inhibitor.

The invention also relates to a method for determining the biological activity of a compound that inhibits the tyrosine kinase activity of the EGFR, which comprises detecting in a mammal to which said EGFR tyrosine kinase inhibitor had been administered the level of expression of a gene the expression level of which correlates with the biological activity of said EGFR tyrosine kinase inhibitor.

The invention further also relates to a method for measuring the biological activity of a compound that inhibits the tyrosine kinase activity of the EGFR, which comprises exposing EGFR expressing cells of a mammal to an EGFR tyrosine kinase inhibitor and detecting the level of expression of a gene the expression level of which correlates with the biological activity of said EGFR tyrosine kinase inhibitor.

In a preferred embodiment of the invention intracellular gene expression levels of a gene the expression level of which correlates with the biological activity of an EGFR tyrosine kinase inhibitor are detected.

In another preferred embodiment of the invention extracellular gene expression levels of a gene the expression level of which correlates with the biological activity of an EGFR tyrosine kinase inhibitor are detected.

In a very preferred embodiment of the process of the present invention, the biological sample has been obtained from a mammal to which said EGFR tyrosine kinase inhibitor had been administered.

The general terms used hereinbefore and hereinafter preferably have the following meanings, if not indicated otherwise:

The term “biological sample” is meant to include any biological material separated from the mammalian body such as e.g. tissue, cell lines, plasma or serum.

Preferably human is meant with the term “mammal” or “mammalian”.

The term “ex vivo” means outside the body of the mammal.

The term “a compound that inhibits the tyrosine kinase activity of the EGFR (=EGFR tyrosine kinase inhibitor)” is understood to mean that in a tyrosine kinase inhibition assay using for example the recombinant intracellular domain of the EGFR [see, for example, E. McGlynn et al., Europ. J. Biochem. 207, 265-275 (1992)] such a compound inhibits the tyrosine kinase activity of the EGFR tyrosine kinase domain by 50% (IC₅₀) in a concentration of less than 10 μM, preferably less than 1 μM, more preferably less than 100 nM, most preferably less than 10 nM. In a more specific sense the term “a compound that inhibits the tyrosine kinase activity of the EGFR” is understood to mean that such a compound exhibits anti-proliferative activity, especially anti-tumor activity.

The term “a gene the expression level of which correlates with the biological activity of an EGFR tyrosine kinase inhibitor” is understood to mean that the level of expression of such a gene is related to the concentration of the administered EGFR tyrosine kinase inhibitor. Such a gene can either be repressed or preferably be induced upon administration of an EGFR tyrosine kinase inhibitor.

If the EGFR tyrosine kinase inhibitor is administered for example to a cell culture, the level of expression of “a gene the expression level of which correlates with the biological activity of an EGFR tyrosine kinase inhibitor” shows a correlation to the concentration of the EGFR tyrosine kinase inhibitor in the cell culture supernatant. If, on the other hand, the EGFR tyrosine kinase inhibitor is administered for example to a mammal, the level of expression of “a gene the expression level of which correlates with the biological activity of an EGFR tyrosine kinase inhibitor” shows a correlation to the concentration of the EGFR tyrosine kinase inhibitor in e.g. the plasma and/or a certain tissue, especially tumor tissue, of such a mammal.

The term “intracellular gene expression level” refers to the level of expression of a gene in a cell, i.e. for example the level of RNA expression or the level of expression of an intracellular protein or a transmembrane protein.

The term “extracellular gene expression level” refers to the level of expression of a gene outside a cell, i.e. for example the level of expression of a secreted extracellular protein.

Preferably, the following is meant by an EGFR tyrosine kinase inhibitor:

(i) a compound of the formula I

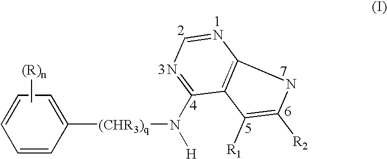

wherein

q is 0 or 1,

n is from 1 to 3 when q is 0, or n is from 0 to 3 when q is 1,

R is halogen, lower alkyl, hydroxy, lower alkanoyloxy, lower alkoxy, carboxy, lower alkoxycarbonyl, carbamoyl, N-lower alkyl-carbamoyl, N,N-di-lower alkyl-carbamoyl, cyano, amino, lower alkanoylamino, lower alkylamino, N,N-di-lower alkylamino or trifluoromethyl, it being possible when several radicals R are present in the molecule for those radicals to be identical or different,

a) R₁ and R₂ are each independently of the other

α) phenyl substituted by carbamoyl-methoxy, carboxy-methoxy, benzyloxycarbonyl-methoxy, lower alkoxycarbonyl-methoxy, phenyl, amino, lower alkanoylamino, lower alkylamino, N,N-di-lower alkylamino, hydroxy, lower alkanoyloxy, carboxy, lower alkoxycarbonyl, carbamoyl, N-lower alkyl-carbamoyl, N,N-di-lower alkyl-carbamoyl, cyano or by nitro;

β) hydrogen;

γ) unsubstituted or halo- or lower alkyl-substituted pyridyl;

δ) N-benzyl-pyridinium-2-yl; naphthyl; cyano; carboxy; lower alkoxycarbonyl; carbamoyl; N-lower alkyl-carbamoyl; N,N-di-lower alkyl-carbamoyl; N-benzyl-carbamoyl; formyl; lower alkanoyl; lower alkenyl; lower alkenyloxy; or

ε) lower alkyl substituted by

εα) halogen, amino, lower alkylamino, piperazino, di-lower alkylamino,

εβ) phenylamino that is unsubstituted or substituted in the phenyl moiety by halogen, lower alkyl, hydroxy, lower alkanoyloxy, lower alkoxy, carboxy, lower alkoxycarbonyl, carbamoyl, N-lower alkyl-carbamoyl, N,N-di-lower alkyl-carbamoyl, cyano, amino, lower alkanoylamino, lower alkylamino, N,N-di-lower alkylamino or by trifluoromethyl,

εγ) hydroxy, lower alkoxy, cyano, carboxy, lower alkoxycarbonyl, carbamoyl, N-lower alkyl-carbamoyl, N,N-di-lower alkyl-carbamoyl, mercapto or

εδ) by a radical of the formula R₄—S(O)ₘ— wherein R₄ is lower alkyl and m is 0, 1 or 2, or

b) when q is 0, one of the radicals R₁ and R₂ is unsubstituted lower alkyl or unsubstituted phenyl and the other of the radicals R₁ and R₂ has one of the meanings given above in paragraph a) with the exception of hydrogen, or

c) R₁ and R₂ together are C₄-C₁₀-1,4-alkadienylene substituted by amino, lower alkanoylamino, lower alkylamino, N,N-di-lower alkylamino, nitro, halogen, hydroxy, lower alkanoyloxy, carboxy, lower alkoxycarbonyl, carbamoyl, N-lower alkyl-carbamoyl, N,N-di-lower alkyl-carbamoyl or by cyano, or are aza-1,4-alkadienylene having up to 9 carbon atoms, or

d) when q is 1, R₁ and R₂ are each independently of the other unsubstituted lower alkyl or unsubstituted phenyl or have one of the meanings given above in paragraph a), and

R₃ is hydrogen, lower alkyl, lower alkoxycarbonyl, carbamoyl, N-lower alkyl-carbamoyl or N,N-di-lower alkyl-carbamoyl,

with the exception of the compound of formula I wherein n is 0, q is 1, R₁ and R₂ are each hydrogen and R₂ is methyl,

(ii) a compound of the formula I wherein

n is 0 to 3,

q is 0 or 1,

R is halogen, lower alkyl, hydroxymethyl, aminomethyl, hydroxy, lower alkanoyloxy, lower alkoxy, carboxy, lower alkanoyl, benzoyl, lower alkoxycarbonyl, carbamoyl, N-lower alkylcarbamoyl, N,N-di-lower alkylcarbamoyl, cyano, amino, lower alkanoylamino, lower alkylamino, N,N-di-lower alkylamino or trifluoromethyl, it being possible, if two or more radicals R are present in the molecule, for these to be identical to or different from one another,

one of the radicals R₁ and R₂ is hydrogen or lower alkyl,

and the other of the radicals R₁ and R₂ is

a) a radical of the formula II

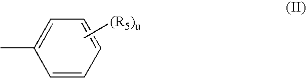

in which u is 1 to 3 and

at least one radical R₅ is amidino, guanidino, ureido, N³-lower alkylureido, N³,N³-di-lower alkylureido, N³-phenylureido, N³,N³-diphenylureido, thiocarbamoyl, thioureido, N³-lower alkylthioureido, N³,N³-di-lower alkylthioureido, lower alkoxycarbonylamino, benzyloxycarbonylamino, morpholine-4-carbonyl, piperazine-1-carbonyl, 4-lower alkylpiperazine-1-carbonyl, lower alkylsulfonylamino, benzenesulfonylamino, toluenesulfonylamino, thiophene-2-carbonylamino, furan-2-carbonylamino, benzylamino, hydroxymethyl, aminomethyl or a radical of the formula —N═C(R₆)—R₇, in which R₆ is hydrogen or lower alkyl and R₇ is di-lower alkylamino, piperidino, 4-lower alkylpiperazino or morpholino, and the other radical(s) R₅ is (are) halogen, lower alkyl, hydroxy, lower alkanoyloxy, lower alkoxy, carboxy, lower alkoxycarbonyl, carbamoyl, N-lower alkylcarbamoyl, N,N-di-lower alkylcarbamoyl, cyano, amino, lower alkanoylamino, lower alkylamino, N,N-di-lower alkylamino or trifluoromethyl, it being possible, if two or more radicals R₅ are present in the molecule, for these to be identical to or different from one another, or is

b) a radical of the formula III

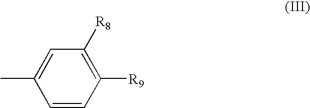

in which R₈ is lower alkoxy or benzyloxy and R₉ is hydroxy or benzyloxy, or is

c) amino-lower alkyl, in which the amino group is substituted by one or two hydroxy-lower alkyl, amino-lower alkyl, carboxy-lower alkyl, lower alkoxycarbonyl-lower alkyl, benzyloxycarbonyl-lower alkyl or benzyl radicals, which in the phenyl moiety are unsubstituted or substituted by halogen, lower alkyl, hydroxymethyl, aminomethyl, hydroxy, lower alkanoyloxy, lower alkoxy, carboxy, lower alkanoyl, benzoyl, lower alkoxycarbonyl, carbamoyl, N-lower alkylcarbamoyl, N,N-di-lower alkylcarbamoyl, cyano, amino, lower alkanoylamino, lower alkylamino, N,N-di-lower alkylamino or trifluoromethyl, or is

d) piperidine-1-carbonyl, piperazine-1-carbonyl, 4-lower alkylpiperazine-1-carbonyl, morpholine-4-carbonyl, thiocarbamoyl, a heterocyclic radical bonded via a ring carbon atom and having five ring members and 1-4 ring heteroatoms, selected from oxygen, nitrogen and sulfur, or is

e) 4-lower alkylpiperazinomethyl or a lower alkyl radical which is substituted by a heterocyclic radical other than piperazinyl and having five or six ring members and 1-4 ring heteroatoms, selected from oxygen, nitrogen and sulfur, or is

f) a radical of the formula —CH═N—OR₁₀ in which R₁₀ is hydrogen or lower alkyl, or

g) if q is 1, additionally to the definitions given above in the sections (ii) a) to f) can also be phenyl which is substituted by halogen, lower alkyl, trifluoromethyl or lower alkoxy, and

R₃ is hydrogen, lower alkyl, lower alkoxycarbonyl, carbamoyl, N-lower alkylcarbamoyl or N,N-di-lower alkylcarbamoyl, or

(iii) a compound of the formula I wherein

q is 0,

n is from 0 to 5,

R is a substituent selected from halogen, lower alkyl, trifluoromethyl and lower alkoxy, and

R₁ and R₂ are each independently of the other lower alkyl, or phenyl that is unsubstituted or substituted by halogen, trifluoromethyl, lower alkyl or by lower alkoxy, it also being possible for one of the two radicals R₁ and R₂ to be hydrogen, or R₁ and R₂ together form an alkylene chain having from 2 to 5 carbon atoms that is unsubstituted or substituted by lower alkyl,

or a salt of such compounds.

More preferably, the following is meant by an EGFR tyrosine kinase inhibitor:

(R)-4-[(1-phenyl-ethyl)-amino]-6-(3-propionylamino-phenyl)-7H-pyrrolo[2,3-d]pyrimidine or a salt thereof.

Most preferably, the following is meant by an EGFR tyrosine kinase inhibitor:

(R)-6-(4-hydroxy-phenyl)-4-[(1-phenyl-ethyl)-amino]-7H-pyrrolo[2,3-d]pyrimidine or a salt thereof.

The EGFR tyrosine kinase inhibitors described hereinabove as being preferred can be prepared for example as described in EP 0 682 027 B1, WO 97/02266 and WO 98/07726.

A salt is preferably a pharmaceutically acceptable salt.

A gene the expression level of which correlates with the biological activity of an EGFR tyrosine kinase inhibitor is preferably a gene mentioned in Tabel 4 below, more preferably the TRAIL, VAC-beta and clusterin gene, most preferably the clusterin gene.

### EXAMPLES

### Materials and Methods

1. Cell Culture

The following tissue culture cells are chosen because of their EGFR expression status (Table 1). EGFR expression is measured by Western blotting, using anti-EGFR monoclonal antibodies from Transduction Labs (Lexington, Ky.). The cell lines are obtained from the ATCC (Rockville, N. Dak.). The cells are cultured in 5% CO₂ at 37° C.

TABLE 1

| | | EGFR | |
|---|---|---|---|
| Name | Description | status | Culture Medium |
| A431 | epidermoid | ++++ | DMEM with 4.5 g/l |
| | carcinoma | | Glucose, 10% FCS |
| NCI-H596 | Adenosquamous | ++ | RPMI 1640 medium, |
| | carcinoma, lung | | 10% FCS |
| NCI-H520 | squamous cell | − | RPMI 1640 medium, |
| | carcinoma, lung | | 10% FCS |
| MDA-MB468 | adenocarcinoma, | ++++ | DMEM with 4.5 g/l |
| | mammary gland | | Glucose, 10% FCS |

The leucocyte-enriched fraction (buffy coat) is obtained from a healthy donor. 10 ml buffy coat, diluted with 25 ml RPMI 1640 medium is layered on 4 ml Ficoll-Paque (Pharmacia) and centrifuged at 1′200×g for 15 min. The White Blood Cell (WBC) layer at the interface is removed, the cells are washed 2 times with RPMI 1640 medium to remove platelets. WBCs are cultured in RPMI 1640 medium plus 10% Fetal Calf Serum (FCS) in T-150 flask, 100 million cells/100 ml and are incubated for 1 h at 37° C. before treatment.

2. Treatment of Cells with an EGFR Tyrosine Kinase Inhibitor and RNA Preparation

2.1. Cells for cDNA Microarray Hybridization:

20 million tissue culture cells (about 80% confluent) or 200 million WBCs are incubated for 2 or 24 h in culture medium containing 3 μM of an EGFR tyrosine kinase inhibitor or 0.03% DMSO. Total RNA from cell pellet is isolated using the RNeasy kit (Qiagen). mRNA is isolated using the oligotex mRNA purification kit (Qiagen). The quality of the obtained mRNA is tested by real time RT-PCR using beta-Actin primers (PE Applied Biosystems) with an amplicon size of 300 base pairs (bp). The total RNA is used as a reference to calculate the amount of mRNA-enrichment in the oligotex purified samples.

2.2. Cells for Real Time RT-PCR:

2 million A431 cells at about 80% confluency are treated with an EGFR tyrosine kinase inhibitor (100, 300 or 3000 nM) or 0.03% DMSO and incubated for 4, 8, 16 or 24 h. Total RNA is isolated using the RNeasy kit.

3. cDNA Array Hybridization

Probes for the UniGEM hybridization are synthesized by Incyte Genomics (Palo Alto, Calif., USA). For each probe 200 ng mRNA (50 ng/μl) is shipped in dry ice. The results are analyzed using the Expressionist Software Package provided by GeneData.

4. Real Time RT-PCR

4.1. Primers Design:

To design primers and probes the primer express software from PE Applied Biosystems is used. The software designed primers with a Melting Temperature (Tm) of 58-60° C. Primer express usually finds up to 200 possible combinations. The best combination showing the following features are chosen manually:

Primers with less than 3 G or Cs within the last five nucleotides at the 3′end.

Amplicon size of less than 150 bp.

The primers and probes are designed from mRNA sequences within the coding region or in the 3′ untranslated region (Table 2).

TABLE 2

| Gene description | Primer name | Primer sequence |
|---|---|---|
| Human complement cytolysis | CLI.forward | 5′-GCTGCAGGAATACCGCAAA-3′ |
| inhibitor (clusterin) | CLI.reverse | 5′-CCGTAGGTGCAAAAGCAACA-3′ |
| Transforming growth factor-beta | BIGH3.forward | 5′-GCTCATAAAACATGAATCAAGCAATC-3′ |
| induced gene product | BIGH3.reverse | 5′-GCTGTGCAAGGGCTTTACAAA-3′ |
| Human TNF-related apoptosis | TRAIL.forward | 5′-TGTTTCTGTAACAAATGAGCACTTGA-3′ |
| inducing ligand TRAIL | TRAIL.reverse | 5′-TTCTTTCCAGGTCAGTTAGCCAA-3′ |
| Human caveolin | CAVEO.forward | 5′-AGCTGAGCGAGAAGCAAGTGT-3′ |
| | CAVEO.reverse | 5′-TGTTTAGGGTCGCGGTTGA-3′ |
| Human acute phase serum | SAAB.forward | 5′-GTGGCAGAGACCCCAATCA-3′ |
| amyloid A protein | SAAB.reverse | 5′-GCAGAGTGAAGAGGAAGCTCAGT-3′ |
| Human extracellular protein (S1-5) | S1-5.forward | 5′-GACATTGTCCCAGACGCTTGT-3′ |
| | S1-5.reverse | 5′-TTTCGGAAGGCAGAGGTATCC-3′ |
| Human annexin VIII (VAC-beta) | VACB.forward | 5′-CACTGGCCCTCCAAGACG-3′ |
| | VACB.reverse | 5′-TCATCTCATCAGTCCCACGAATC-3′ |
| Human cathepsin H | CATHH.forward | 5′-GCAACCGGAAAGATGCTGTC-3′ |
| | CATHH.reverse | 5′-CCCTTGGCAGCCGTAATTATT-3′ |
| Human complement component 3 (CS) | C3.forward | 5′-AGATAAGAACCGCTGGGAGGA-3′ |
| | C3.reverse | 5′-GGGCCAAGAGGGCATAGG-3′ |
| DNA-binding protein inhibitor ID-3 | HLH1R2.forward | 5′-GAACTGGTACCCGGAGTCCC-3′ |
| | HLH1R2.reverse | 5′-CGATGACGCGCTGTAGGAT-3′ |
| endogenous reference | GAPDH.forward | 5′-GCACCGTCAAGGCTGAGAAC-3′ |
| | GAPDH.reverse | 5′-GAGGGATCTCGCTCCTGGA-3′ |

4.2. RT-PCR Reaction:

For real time RT-PCR, RNA concentrations between 1 and 50 ng/reaction are used to obtain signals between 15-35 cycles. For each RNA preparation RT-PCR reactions are performed with and without reverse transcriptase to check for genomic DNA contamination. In all RNA samples tested, genomic DNA contamination is less than 1%. To normalize for variability in the initial concentration and quality of the total RNA, Glyceraldehyde-3-phosphate dehydrogenase (GAPDH) is used as an endogenous reference in all quantification experiments.

For RT-PCR a one step, two-enzyme system is chosen. The TaqMan PCR Core Reagent Kit (PE #N808-0228) plus the reverse transcriptase MuLV (PE Biosystems # N808-0018) and RNase inhibitor (Roche Diagnostics # 799 025) are mixed as described in the manufacturers protocol. Triplicate measurements are done for target and for reference reactions.

### Thermal Cycling Conditions

| | | | |
|---|---|---|---|
| RT step | 48° C. | 30 min | |
| Ampli Taq Gold activation | 95° C. | 10 min | |
| PCR step | 95° C. | 15 sec | 40 cycles |
| | 60° C. | 1 min | |

4.3. Optimization of Primers and Probe:

Primers and probe for each gene to be tested are optimized using the following scheme. Final concentrations are given:

| | | | | | | | | | |
|---|---|---|---|---|---|---|---|---|---|
| forward | 100 | 100 | 100 | 300 | 300 | 300 | 900 | 900 | 900 |
| primer | | | | | | | | | |
| (nM): | | | | | | | | | |
| reverse | 100 | 300 | 900 | 100 | 300 | 900 | 100 | 300 | 900 |
| primer | | | | | | | | | |
| (nM): | | | | | | | | | |

As the optimal concentration, the reaction with the lowest C_{T} value in first priority and with the highest Rn (relative fluorescence minus background fluorescence) value in second priority is chosen.

4.4. Comparative C_{T} Method:

Before using the comparative C_{T} Method for quantification, a validation experiment is performed to demonstrate that efficiencies of target and reference are approximately equal. RT-PCR reactions are done with RNA concentrations of 2.5, 5, 10, 20, 40 and 80 ng per reaction. The absolute value of the slope of (log input amount) vs. ΔC_{T} should be <0.1. The ΔC_{T} value is determined by subtracting the average reference C_{T} values from the average target C_{T} values of the three measurements. Relative quantification is performed in triplicates, reference and target, using the RT-PCR protocol described above. Results are obtained using the following calculation:

| | |
|---|---|
| ΔC_{T}: | C_{T} target − C_{T} reference |
| s: | standard deviation of the difference s = square root (S₁² + S₂²) |
| ΔΔC_{T}: | ΔC_{T} − ΔC_{T} calibrator (This is subtraction of an arbitrary |
| | constant. Therefore the standard deviation of ΔΔC_{T} is the same |
| | as the standard deviation of ΔC_{T}). |
| range: | The range given for a target relative to the calibrator is |
| | determined by evaluating the expression 2^{-ΔΔCT} with ΔΔ^{CT+s} and |
| | ΔΔ^{CT-s}. |

5. Western Blot Analysis

5.1. Treatment of Cells with an EGFR Tyrosine Kinase Inhibitor or DMSO:

An EGFR tyrosine kinase inhibitor is dissolved in DMSO to obtain a 10 mM stock solution. The stock solution is further diluted in culture medium to final concentrations of 10 nM to 3 μM.

3.5 million cells/100 mm dish/12 ml culture medium are incubated for 24 h in 5% CO₂ at 37° C. The medium is replaced with medium containing the EGFR tyrosine kinase inhibitor or DMSO. Cells are incubated for 4 to 32 h.

After incubation, the cells are trypsinized and washed once with Phosphate Buffered Saline (PBS). The cell pellets are frozen at −80° C.

5.2. Cell Lysis:

For the initial experiments, cells are lysed in M-PER mammalian protein reagent (PIERCE). Due to protein precipitation and/or aggregation the procedure is changed. The following method yields reproducible results:

The frozen pellets are lysed in 500 μl of lysis buffer (50 mM Tris-HCl pH 7.5, 1% Sodium Dodecyl Sulfate (SDS)).

To shear genomic DNA, the cell lysates are processed in a FastPrep instrument for 20 sec at a speed rating of 4, using the green FastRNA tubes (Bio101#6040-6010). Protein concentrations are determined according to the PIERCE-method (BCA Protein Assay, Reagent A and B). Protein concentration of lysates is adjusted to 1 μg/μl with lysis buffer. 4 parts of cell lysates are mixed with 1 part of 5 fold concentrated Laemmli buffer, the lysates are stored at −20° C.

5.3. SDS-Polyacrylamide Gel Electrophoresis (SDS-PAGE), Blotting and Detection:

40 μl of cell lysate is heated for 3 min to 95° C. and directly loaded on to 10% or 15% SDS-PAGE gels. The gels are run at 130 V for 90 min under standard conditions. The separated proteins are semi-dry blotted on to a PVDF membrane (Immobilon-P, Millipore #IPVH 202 00) for 70 min with 45 mA per blot.

Immediately after the transfer the blots are incubated in Blotto A (5% skim milk powder in Tris Buffered Saline (TBS)/0.05% Tween-20) for 1 h at room temperature on a rotating platform. The blots are then washed three times in TBS/0.05% Tween-20. The blots are incubated for 1 h with 0.2 μg/ml of e.g. goat anti-human clusterin (C-18) antibody (Santa Cruz, sc-6419) in Blotto A for 1.5 h at room temperature on a rotating platform. The control blots are incubated with 0.2 μg/ml of mouse anti-human-actin (C-2) antibody (Santa Cruz, sc-8432). The blots are washed three times with TBS/0.05% Tween-20. The blots are incubated with the secondary anti-sheep/goat alkaline-phospatase conjugated antibody (Roche), or secondary anti-mouse alkaline-phospatase conjugated antibody (Santa Cruz sc-2302) diluted 1:1000 with Blotto A, for 1 h at room temperature. Surplus antibody is removed in three washes (TBS/0.05% Tween-20). The blots are carefully overlaid with 0.125 ml/cm² (approximately 1.5 ml per blot) SuperSignal® chemiluminescent-substrate (PIERCE) for 1 min. Excess reagent is removed and the blots are covered with transparent plastic wrap. The blots are exposed to X-ray films (CL-XPosure, PIERCE) for 1 to 10 min.

### EXAMPLES

### Example 1

Expression profiles (UniGEM V arrays) of 4 tumor cell lines with different EGFR expression status (A431, NCI-H596, NCI-H520 and MDA-MB-468) and of WBCs upon exposure of cells to 3 μM of COMPOUND A for 2 or 24 h are analyzed. As control, cells treated with DMSO for the same time period are used. The 2 h expression profiles, with a cut off set at 3, shows almost no induced genes and only few repressed genes, e.g. early growth response protein 1, which shows repression in two cell lines and WBCs (data not shown). From the 24 h expression profiles, the A431 cell line shows the most promising results, with several differentially expressed genes. The cDNA array hybridization is repeated with the UniGEM 1 array using the same mRNA (Table 3).

TABLE 3
UniGEM 1 cDNA array expression profile of A431 cells treated with
3 μM of COMPOUND A for 24 hours, versus DMSO treated cells

| | | Differential |
|---|---|---|
| | | Expression |
| GB Acc. No. | Gene Description | (Balanced) |
| W46413 | DNA-BINDING PROTEIN INHIBITOR | 2.1 |
| | ID-3 {IM323946} | |
| AA057802 | TransformIng growth factor-beta induced | 2.1 |
| | gene product (BIGH3) | |
| AA058337 | Human mRNA for cathepsin H | 2.2 |
| | (E.C.3.4.22.1) {IM489348} | |
| AA025750 | Human HE4 mRNA for extracellular | 2.6 |
| | protease {IM366323} | |
| R39436 | ESTs {IM23605} | 2.6 |
| N53767 | ESTs {248032} | 2.6 |
| T54672 | ESTs {IM73785} | 2.6 |
| AA057780 | ESTs {IM377004} | 2.7 |
| AA025124 | ESTs {IM365120} | 2.7 |
| M25915 | Human complement cytolysis inhibitor | 2.7 |
| | (CLI) mRNA; complete cds (Clusterin) | |
| AA026089 | Human mRNA for precursor of epidermal | 2.8 |
| | growth factor receptor {IM469272} | |
| AA029438 | Human cyclin-dependent protein kinase | 2.8 |
| | mRNA {IM366824} | |
| R13994 | Rat N-syndecan mRNA {IM26677} | 2.9 |
| T59122 | ESTs {IM74546} | 2.9 |
| AA041250 | Human mRNA for pancreatic carcinoma | 3.0 |
| | marker {IM376296} (Trop-2) | |
| AA046102 | Human annexin VIII mRNA; complete cds. | 3.0 |
| | {IM376634} (VAC-beta) | |
| AA035156 | ESTs {IM471869} | 3.0 |
| AA041549 | Human complement component 3 (C3) | 3.1 |
| | gene {IM485717} | |
| AA030000 | Human extracellular protein (S1-5) mRNA | 3.3 |
| | {IM470007} | |
| AA047243 | Human mRNA for caveolin {IM488533} | 3.4 |
| H54629 | Human TNF-related apoptosis inducing | 4.0 |
| | ligand TRAIL {IM203132} | |
| H25546 | Human acute phase serum amyloid A pro- | 4.4 |
| | tein {IM161456} | |
| H09077 | ESTs {IM46265} | −21.9 |
| AA057694 | Fibronectin {IM512287} | −8.8 |
| AA010487 | Human D-type cyclin (CCND2) mRNA | −8.2 |
| | {IM359412} | |
| H03907 | Human fibronectin gene extra type III re | −6.9 |
| | {IM151144} | |
| AA039628 | ESTs {IM485336} contains Alu repetitive | −6.8 |
| | elements | |
| R36450 | Human fibronectin (fn) 3′ coding region | −4.7 |
| | {IM136798} | |
| AA040600 | Human 5′-AMP-activated protein kinase; g | −4.1 |
| | {IM376178} | |
| H17614 | Human ribosomal protein L5 pseudogene | −3.6 |
| | mRNA {IM50482} | |
| AA039353 | Human nucleotide-binding protein mRNA; c | −3.5 |
| | {IM376052} | |
| T52813 | PUTATIVE LYMPHOCYTE G0/G1 | −3.3 |
| | SWITCH PROTEIN 2 {IM67939} | |
| H18621 | ESTs {IM172005} | −3.1 |
| W95433 | Human nuclear orphan receptor LXR-alpha | −3.0 |
| | {IM357775} | |
| T76993 | Human mitosin mRNA; complete cds. | −3.0 |
| | {IM113765} | |

positive numbers: induced genes, cut off = 2.0; genes selected for confirmation with real time RT-PCR are in bold.
negative numbers: repressed genes, cut off = −3.
GB Acc. No.: Gene Bank Accession Number;
EST: expressed sequence tag.

The two cDNA array hybridizations show good correlation (Table 4). Ten genes are selected for confirmation by real time RT-PCR, based on the following criteria: consistent regulation in both cDNA array experiments, induced but not repressed genes, good signal to background ratio (>5), preferentially genes that encode for cell surface or secreted proteins and genes that have been linked to apoptosis or growth control. The induction upon COMPOUND A exposure can be confirmed for all of the selected genes, when testing the same RNA that is used for cDNA array hybridization (Table 4).

TABLE 4
Gene induction upon exposure of A431 cells to 3 μM of COMPOUND A
for 24 hours, confirmed by real time RT-PCR

| | Micro Array | | | | |
|---|---|---|---|---|---|
| | RNA from Experiment | | Real time RT-PCR | | |
| | 1 | | Experiment | Experiment | Experiment |
| Gene Description | UniGEM 1 | UniGEM V | 1 | 2 | 3 |
| Complement cytolysis inibitor | 2.7 | 6.6 | 11.2 ± 2.0 | 5.2 ± 1.4 | 6.2 |
| (Clusterin) | | | | | |
| TRAIL | 3.0 | 4.0 | 7.8 ± 1.6 | 4.1 ± 1.4 | 5.7 ± 0.03 |
| Extracellular protein S1-5 | 3.3 | 2.2 | 3.2 ± 1.7 | 1.7 ± 0.2 | 1.2 ± 0.5 |
| TGF-beta induced gene | 2.1 | 2.7 | 5.2 | nt | nt |
| DNA binding protein inhibitor ID-3 | 2.1 | 2.6 | 4.4 | 2.2 ± 0.1 | 2.4 |
| Acute phase serum amyloid A | 4.4 | 3.3 | 6.6 | 2.5 ± 1.1 | 3.3 |
| protein | | | | | |
| Cathepsin H | 2.2 | 2.4 | 5.2 | 2.6 ± 0.9 | 2.1 |
| Caveolin | 3.4 | np | 2.8 ± 0.2 | 1.5 ± 0.4 | 2.6 |
| VAC-beta (annexin VIII) | 3.0 | 2.2 | 2.8 ± 1.5 | 2.4 ± 0.9 | 2.5 ± 0.8 |
| Complement component 3 (C3) | 3.1 | 3.3 | 5.6 | 2.5 ± 0.4 | 2.0 |

Experiments 1, 2 and 3 represent completely independent, but identical experiments. Average and standard deviations (n = 2-5) were calculated when more than one real time RT-PCR experiment was done.
np: not present;
nt: not tested because of very low expression levels.

The treatment of A431 cells with 3 μM of COMPOUND A is repeated twice in independent experiments to confirm the induction of gene expression levels. The induction upon COMPOUND A exposure can be repeated for all genes, except for human extracellular protein S1-5 (Table 4).

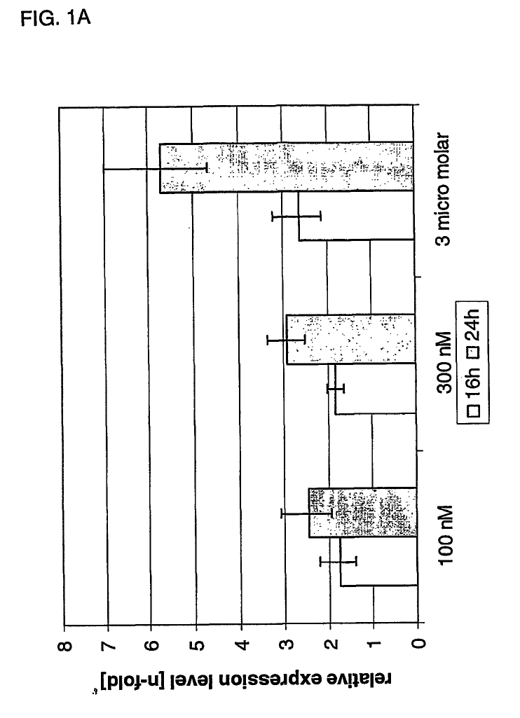

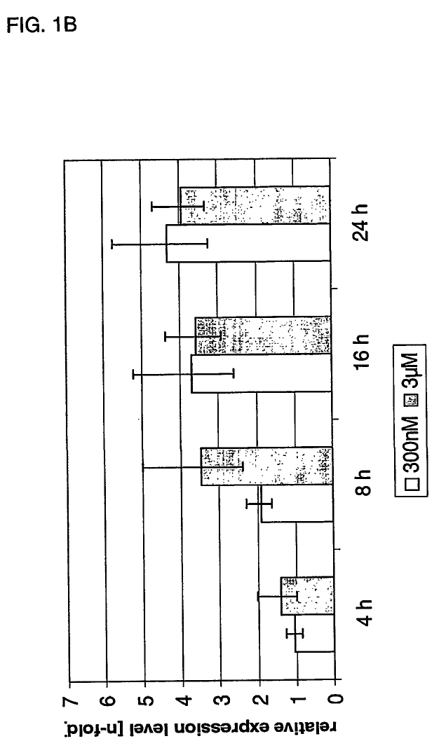

The induction of gene expression shows dose and time dependency as d monstrated for clusterin (FIGS. 1A and 1B).

Expression levels for TRAIL, clusterin and VAC-beta are tested with real time RT-PCR in all cell lines and WBCs treated for 24 h with 3 μM of COMPOUND A or with DMSO. Expression levels for the three genes, calculated relative to NCI-H520, show big differences between the various cell types (Table 5). NCI-H520 is designated as the calibrator, because of the low expression level of all three genes in this cell line. The expression levels of VAC-beta reach the detection limit in NCI-H520 cells and WBCs. Differential expression upon COMPOUND A treatment is consistent with cDNA array hybridization (UniGEM V) results in all tested cell lines and WBCs. In contrast to A431 cells, TRAIL shows a relatively high basal expression level in WBCs and a 2.5 fold repression after COMPOUND A exposure for 24 h (Table 5).

TABLE 5
Relative TRAIL, Clusterin and VAC-beta expression
in various cell lines

| | TRAIL | | Clusterin | | VAC-beta | |
|---|---|---|---|---|---|---|
| | | 3 μM | | 3 μM | | 3 μM |
| Cell | | Com- | | Com- | | Com- |
| Line | DMSO | pound A | DMSO | pound A | DMSO | pound A |
| A431 | 11.16 | 99.50 | 21.46 | 247.28 | 13.00 | 57.20 |
| | (10.32- | (85.98- | (19.79- | (231.35- | (11.00- | (55.27- |
| | 12.07) | 115.10) | 23.27) | 264.31) | 15.35) | 59.19) |
| MDA- | 2.11 | 2.89 | 10.93 | 32.60 | 4.40 | 26.60 |
| MB468 | (1.59- | (2.65- | (10.56- | (30.25- | (3.80- | (24.46- |
| | 2.80) | 3.14) | 11.31) | 35.13) | 5.09) | 28.93) |
| NCI- | 62.54 | 81.76 | 38.59 | 44.12 | 50.04 | 102.30 |
| H596 | (55.19- | (71.71- | (36.50- | (42.58- | (46.04- | (82.41- |
| | 70.87) | 93.22) | 40.79) | 45.72) | 54.39) | 126.86) |
| NCI- | 1.00 | 0.39 | 1.00 | 0.61 | 1.00 | 0.75 |
| H520 | (0.79- | (0.32- | (0.94- | (0.52- | (0.85- | (0.60- |
| | 1.16) | 0.48) | 1.06) | 0.72) | 1.18) | 0.94) |
| WBC | 57.55 | 22.73 | 1.65 | 2.03 | 1.00 | 2.16 |
| | (52.51- | (20.7- | (1.53- | (1.51- | (0.93- | (1.71- |
| | 63.06) | 24.97) | 1.79) | 2.94) | 1.08) | 2.74) |

Relative [n-fold] TRAIL, clusterin and VAC-beta expression levels measured by real time RT-PCR in various cell lines after 24 h treatment with COMPOUND A or DMSO.
NCI-H520 is used as a calibrator, i.e. a sample used as the basis for comparative analysis of expression levels between independent samples, because of the low expression levels of all three

Relative [n-fold] TRAIL, clusterin and VAC-beta expression levels measured by real time RT-PCR in various cell lines after 24 h treatment with COMPOUND A or DMSO.

NCI-H520 is used as a calibrator, i.e. a sample used as the basis for comparative analysis of expression levels between independent samples, because of the low expression levels of all three genes in this cell line. Ranges given in parenthesis are calculated as described above in Materials and Methods under 4.4. (n=3).

### Example 2

Dose Response of Clusterin Induction in A431 Cells Upon COMPOUND A Exposure

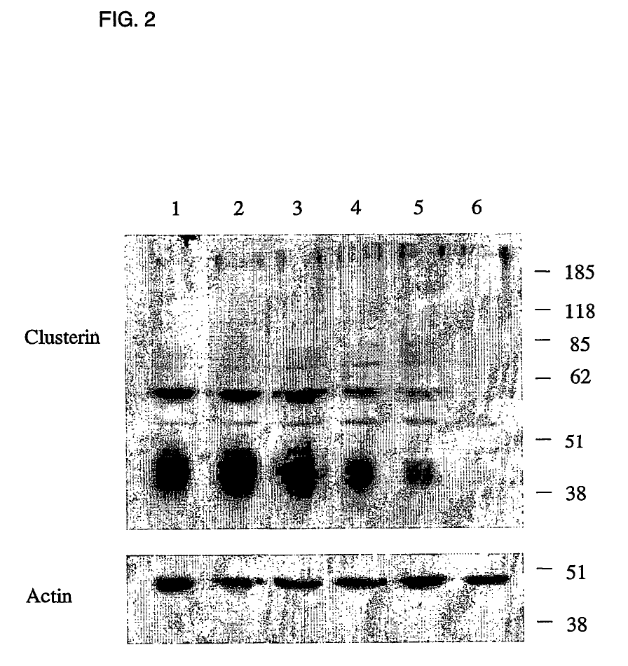

The induction of clusterin (also called pg80, apolipoprotein J, complement cytolysis inhibitor or TRPM-2; see also Jenne D. E. and Tschopp, J., Proc. Natl. Acad. Sci. U.S.A. 86, 7123-7127, 1989) in A431 cells upon exposure of cells to various concentrations of COMPOUND A for 24 h is analyzed. As a control, A431 cells are treated with 0.03% DMSO. FIG. 2 shows the induction of clusterin protein after 24 h treatment with increasing concentrations of COMPOUND A. A low basal level expression of clusterin can be detected. The strong signals seen at approximately 43 kDa (Reddy et al., Biochemistry 35, 6157-6163, 1996) increase with COMPOUND A concentration and reach a maximum with 300 nM. The additional signals which are detected with the anti-clusterin antibody represent most probably glycosylated forms of clusterin precursor (56-60 kDa and 75-80 kDa; Reddy et al., Biochemistry 35, 6157-6163, 1996). The control blot, incubated with anti-actin antibody confirms that equal amounts of protein are loaded per lane.

### Example 3

Time Course of Clusterin Induction in A431 Cells Upon COMPOUND A Exposure

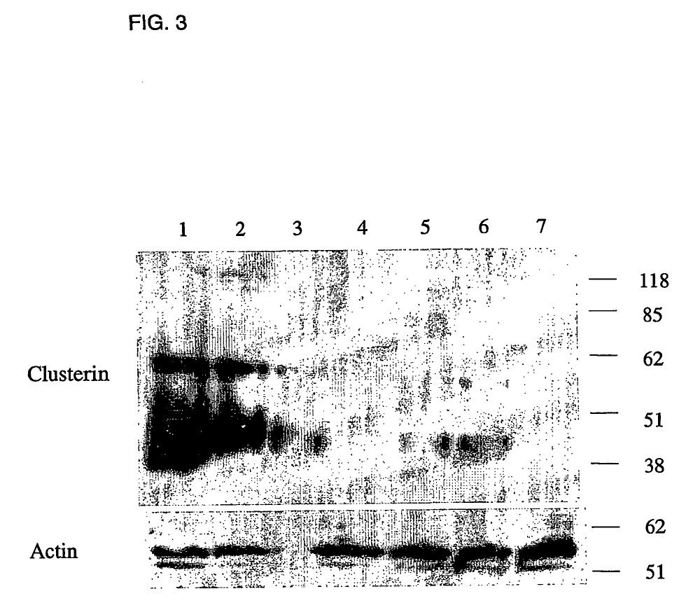

Clusterin protein induction in A431 cells treated with 300 nM of COMPOUND A for 4, 8, 16, 24 and 32 h or with 0.03% DMSO for 4 and 24 h are represented in FIG. 3. A continuous increase of clusterin signal from 16 h to 32 h is detected.

### Example 4

Relationship Between Clusterin Induction by COMPOUND A and EGFR Status

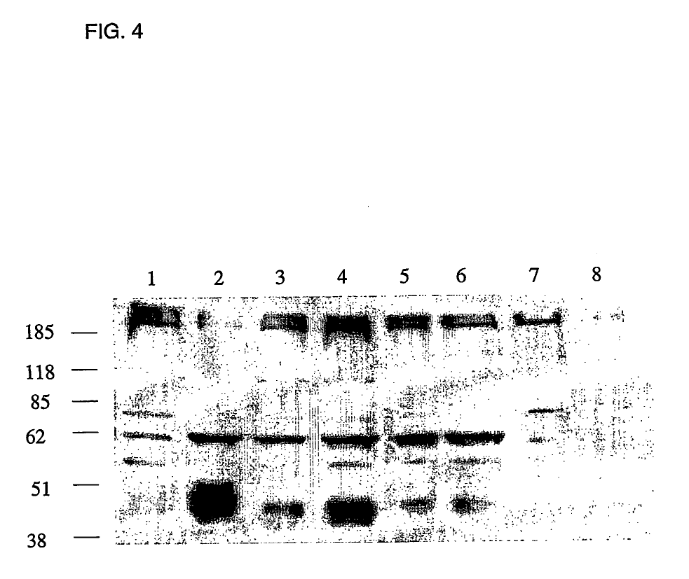

To demonstrate a relationship between EGFR status and clusterin induction, four different cell lines are treated with either 300 nM of COMPOUND A or 0.03% DMSO for 24 h. As shown in FIG. 4, strong clusterin expression signals upon COMPOUND A treatment are detected in A431 and MDA-MB468 cells, both expressing high levels of EGFR. NCI-H596 cells with moderate levels of EGFR show no significant clusterin induction by COMPOUND A. NCI-H520 cells, which do not express EGFR, show no detectable clusterin levels.

What is claimed is:

1. A process for determining the biological activity of a compound that inhibits the tyrosine kinase activity of the epidermal growth factor receptor (EGFR), which process comprises detecting in a biological sample the level of expression of a gene the expression level of which correlates with the biological activity of said EGFR tyrosine kinase inhibitor, said biological sample having been exposed to said EGFR tyrosine kinase inhibitor.

2. A process according to claim 1, wherein the anti-proliferative activity of an EGFR tyrosine kinase inhibitor is determined.

3. A process according to claim 1 or 2, which comprises detecting intracellular gene expression levels.

4. A process according to claim 1 or 2, which comprises detecting extracellular gene expression levels.

5. A process according to any one of claims 1 to 4, which comprises detecting the expression level of the clusterin gene.

6. A process according to any one of claims 1 to 5, wherein the EGFR tyrosine kinase inhibitor is selected from the group consisting of
(i) a compound of the formula I 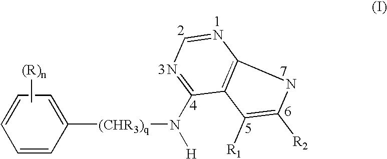
wherein
q is 0 or 1,
n is from 1 to 3 when q is 0, or n is from 0 to 3 when q is 1,
R is halogen, lower alkyl, hydroxy, lower alkanoyloxy, lower alkoxy, carboxy, lower alkoxycarbonyl, carbamoyl, N-lower alkyl-carbamoyl, N,N-di-lower alkyl-carbamoyl, cyano, amino, lower alkanoylamino, lower alkylamino, N,N-di-lower alkylamino or trifluoromethyl, it being possible when several radicals R are present in the molecule for those radicals to be identical or different,
a) R₁ and R₂ are each independently of the other
α) phenyl substituted by carbamoyl-methoxy, carboxy-methoxy, benzyloxycarbonyl-methoxy, lower alkoxycarbonyl-methoxy, phenyl, amino, lower alkanoylamino, lower alkylamino, N,N-di-lower alkylamino, hydroxy, lower alkanoyloxy, carboxy, lower alkoxycarbonyl, carbamoyl, N-lower alkyl-carbamoyl, N,N-di-lower alkyl-carbamoyl, cyano or by nitro;
β) hydrogen;
γ) unsubstituted or halo- or lower alkyl-substituted pyridyl;
δ) N-benzyl-pyridinium-2-yl; naphthyl; cyano; carboxy; lower alkoxycarbonyl; carbamoyl; N-lower alkyl-carbamoyl; N,N-di-lower alkyl-carbamoyl; N-benzyl-carbamoyl; formyl; lower alkanoyl; lower alkenyl; lower alkenyloxy; or
ε) lower alkyl substituted by
εα) halogen, amino, lower alkylamino, piperazino, di-lower alkylamino,
εβ) phenylamino that is unsubstituted or substituted in the phenyl moiety by halogen, lower alkyl, hydroxy, lower alkanoyloxy, lower alkoxy, carboxy, lower alkoxycarbonyl, carbamoyl, N-lower alkyl-carbamoyl, N,N-di-lower alkyl-carbamoyl, cyano, amino, lower alkanoylamino, lower alkylamino, N,N-di-lower alkylamino or by trifluoromethyl,
εγ) hydroxy, lower alkoxy, cyano, carboxy, lower alkoxycarbonyl, carbamoyl, N-lower alkyl-carbamoyl, N,N-di-lower alkyl-carbamoyl, mercapto or
εδ) by a radical of the formula R₄—S(O)ₘ— wherein R₄ is lower alkyl and m is 0, 1 or 2, or
b) when q is 0, one of the radicals R₁ and R₂ is unsubstituted lower alkyl or unsubstituted phenyl and the other of the radicals R₁ and R₂ has one of the meanings given above in paragraph a) with the exception of hydrogen, or
c) R₁ and R₂ together are C₄-C₁₀-1,4-alkadienylene substituted by amino, lower alkanoylamino, lower alkylamino, N,N-di-lower alkylamino, nitro, halogen, hydroxy, lower alkanoyloxy, carboxy, lower alkoxycarbonyl, carbamoyl, N-lower alkyl-carbamoyl, N,N-di-lower alkyl-carbamoyl or by cyano, or are aza-1,4-alkadienylene having up to 9 carbon atoms, or
d) when q is 1, R₁ and R₂ are each independently of the other unsubstituted lower alkyl or unsubstituted phenyl or have one of the meanings given above in paragraph a), and
R₃ is hydrogen, lower alkyl, lower alkoxycarbonyl, carbamoyl, N-lower alkyl-carbamoyl or N,N-di-lower alkyl-carbamoyl,
with the exception of the compound of formula I wherein n is 0, q is 1, R₁ and R₃ are each hydrogen and R₂ is methyl,
(ii) a compound of the formula I wherein
n is 0 to 3,
q is 0 or 1,
R is halogen, lower alkyl, hydroxymethyl, aminomethyl, hydroxy, lower alkanoyloxy, lower alkoxy, carboxy, lower alkanoyl, benzoyl, lower alkoxycarbonyl, carbamoyl, N-lower alkylcarbamoyl, N,N-di-lower alkylcarbamoyl, cyano, amino, lower alkanoylamino, lower alkylamino, N,N-di-lower alkylamino or trifluoromethyl, it being possible, if two or more radicals R are present in the molecule, for these to be identical to or different from one another,
one of the radicals R₁ and R₂ is hydrogen or lower alkyl,
and the other of the radicals R₁ and R₂ is
a) a radical of the formula II 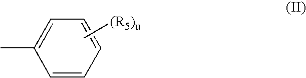
in which u is 1 to 3 and
at least one radical R₅ is amidino, guanidino, ureido, N³-lower alkylureido, N³,N³-di-lower alkylureido, N³-phenylureido, N³,N³-diphenylureido, thiocarbamoyl, thioureido, N³-lower alkylthioureido, N³,N³-di-lower alkylthioureido, lower alkoxycarbonylamino, benzyloxycarbonylamino, morpholine-4-carbonyl, piperazine-1-carbonyl, 4-lower alkylpiperazine-1-carbonyl, lower alkylsulfonylamino, benzenesulfonylamino, toluenesulfonylamino, thiophene-2-carbonylamino, furan-2-carbonylamino, benzylamino, hydroxymethyl, aminomethyl or a radical of the formula —N═C(R₆)—R₇, in which R₆ is hydrogen or lower alkyl and R₇ is di-lower alkylamino, piperidino, 4-lower alkylpiperazino or morpholino, and the other radical(s) R₅ is (are) halogen, lower alkyl, hydroxy, lower alkanoyloxy, lower alkoxy, carboxy, lower alkoxycarbonyl, carbamoyl, N-lower alkylcarbamoyl, N,N-di-lower alkylcarbamoyl, cyano, amino, lower alkanoylamino, lower alkylamino, N,N-di-lower alkylamino or trifluoromethyl, it being possible, if two or more radicals R₅ are present in the molecule, for these to be identical to or different from one another, or is
b) a radical of the formula III 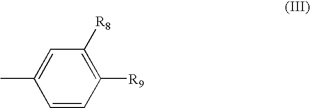
in which R₈ is lower alkoxy or benzyloxy and R₉ is hydroxy or benzyloxy, or is
c) amino-lower alkyl, in which the amino group is substituted by one or two hydroxy-lower alkyl, amino-lower alkyl, carboxy-lower alkyl, lower alkoxycarbonyl-lower alkyl, benzyloxycarbonyl-lower alkyl or benzyl radicals, which in the phenyl moiety are unsubstituted or substituted by halogen, lower alkyl, hydroxymethyl, aminomethyl, hydroxy, lower alkanoyloxy, lower alkoxy, carboxy, lower alkanoyl, benzoyl, lower alkoxycarbonyl, carbamoyl, N-lower alkylcarbamoyl, N,N-di-lower alkylcarbamoyl, cyano, amino, lower alkanoylamino, lower alkylamino, N,N-di-lower alkylamino or trifluoromethyl, or is
d) piperidine-1-carbonyl, piperazine-1-carbonyl, 4-lower alkylpiperazine-1-carbonyl, morpholine-4-carbonyl, thiocarbamoyl, a heterocyclic radical bonded via a ring carbon atom and having five ring members and 1-4 ring heteroatoms, selected from oxygen, nitrogen and sulfur, or is
e) 4-lower alkylpiperazinomethyl or a lower alkyl radical which is substituted by a heterocyclic radical other than piperazinyl and having five or six ring members and 1-4 ring heteroatoms, selected from oxygen, nitrogen and sulfur, or is
f) a radical of the formula —CH|N—OR₁₀ in which R₁₀ is hydrogen or lower alkyl, or
g) if q is 1, additionally to the definitions given above in the sections (ii) a) to f) can also be phenyl which is substituted by halogen, lower alkyl, trifluoromethyl or lower alkoxy, and
R₃ is hydrogen, lower alkyl, lower alkoxycarbonyl, carbamoyl, N-lower alkylcarbamoyl or N,N-di-lower alkylcarbamoyl, and
(iii) a compound of the formula I wherein
q is 0,
n is from 0 to 5,
R is a substituent selected from halogen, lower alkyl, trifluoromethyl and lower alkoxy, and
R₁ and R₂ are each independently of the other lower alkyl, or phenyl that is unsubstituted or substituted by halogen, trifluoromethyl, lower alkyl or by lower alkoxy, it also being possible for one of the two radicals R₁ and R₂ to be hydrogen, or R₁ and R₂ together form an alkylene chain having from 2 to 5 carbon atoms that is unsubstituted or substituted by lower alkyl,
or a salt of such compounds.

7. A process according to claim 6, wherein the EGFR tyrosine kinase inhibitor is (R)-6-(4-hydroxy-phenyl)4-[(1-phenyl-ethyl)-amino]-7H-pyrrolo[2,3-d]pyrimidine or a salt thereof.

8. A process according to claim 6, wherein the EGFR tyrosine kinase inhibitor is (R)-4-[(1-phenyl-ethyl)-amino]-6-(3-propionylamino-phenyl)-7H-pyrrolo[2,3-d]pyrimidine or a salt thereof.

9. A process according to any one of claims 1 to 8, wherein the biological sample has been obtained from a mammal to which said EGFR tyrosine kinase inhibitor had been administered.

10. A process according to claim 9, wherein said mammal is a human.

11. The use of a transcription or translation product of a gene the expression level of which in a mammalian cell correlates with the biological activity of an EGFR tyrosine kinase inhibitor for determining ex vivo the biological activity of such an EGFR tyrosine kinase inhibitor.

12. The use according to claim 11, wherein the gene is the clusterin gene.

13. The use according to claim 11 or 12, wherein the EGFR tyrosine kinase inhibitor is a compound of formula I, wherein the radicals and symbols have the meanings as defined in claim 6, or a salt thereof.

14. A method for determining the biological activity of a compound that inhibits the tyrosine kinase activity of the EGFR, which comprises detecting in a mammal to which said EGFR tyrosine kinase inhibitor had been administered the level of expression of a gene the expression level of which correlates with the biological activity of said EGFR tyrosine kinase inhibitor.
